(19) United States
(12) Patent Application Publication
(10) Pub. No.: US 2007/0036845 A1
(43) Pub. Date: Feb. 15, 2007

(54) Packeted body lotion towelette

(76) Inventors: Ellen D. Persefeld, Garland, TX (US); Lisa Baker, Garland, TX (US)

Correspondence Address: Alan R. Thiele;Strasburger & Price, LLP, Suite 4400, 901 Main St., Dallas, TX, 75202, US

(21) Appl. No.: 11/498,385

(22) Filed: Aug. 3, 2006

Related U.S. Application Data
(60) Provisional application No. 60/708,409, filed on Aug. 15, 2005.

(51) Int. Cl. A61K 9/70; B32B 27/04

(52) U.S. Cl. 424/443; 442/123

(57) ABSTRACT

A packeted towelette impregnated with body lotion is disclosed in the instant application. The towelette is packaged in a leak proof packet and fits easily into a pocket, purse, suitcase, briefcase, golf bag, gym bag or other container. The body lotion contained within the packet is a mixture of many ingredients which, when placed on the skin, helps moisturize and protect the skin from the ravages of the environment.

### CROSS REFERENCE TO RELATED APPLICATIONS

This Utility Patent Application makes reference and seeks priority to Provisional Application No. 60/708,409 filed Aug. 15, 2005.

### STATEMENT REGARDING FEDERALLY SPONSORED RESEARCH OR DEVELOPMENT

The invention described in the instant application was not the subject of federally sponsored research or development.

### FIELD

The present invention pertains to towelettes; more particularly, the present invention pertains to towelettes impregnated with body lotion contained in a single convenient packet.

### BACKGROUND

The skin of human beings acts as an important barrier to protect inner organs from the ravages of the environment. Just as wind and water erodes the soil and breaks down rock to form canyons, these environmental forces and others are also continually wearing down the protective fabric of the skin. Maintaining the integrity of this barrier made of skin is important to protect the human body from infection and to provide a stable environment in which the life processes can function. One of the most convenient ways human beings have chosen to protect the skin is to wear clothes. However, not all parts of the body can be covered by clothes either for the sake of convenience such as operation of the senses, i.e. sight, hearing, speaking, but also for cultural and fashion reasons. As a result, the skin of a human being is constantly exposed to the rigors of the environment and is always in need of protection and/or repair.

### DESCRIPTION OF THE PRIOR ART

The skin-care industry in the U.S. offers a wide variety of products to assist users with a broad array of problems. Such skin-care products include products which act as deodorants, fragrances, moisturizers, sunscreens, cleansers, wrinkle removers, and skin darkeners (tanning lotions). These skin-care products are generally provided to users in bottles, tubes or jars. Some manufacturers provide single-use amounts of some of these products on towelettes.

Examples of products placed on single-use towelettes may be found in the following U.S. patents:

U.S. Pat. No. 6,294,182, describing a towelette for use with a product for minimizing face lines and wrinkles;

U.S. Pat. No. 5,972,360, describing a towelette for use with tanning lotion;

U.S. Pat. No. 5,915,394, describing a towelette for use with fragrances;

U.S. Pat. No. 5,753,246, describing a hand-cleansing towelette; and

U.S. Pat. No. 4,817,790, describing a towelette impregnated with wet powder.

Missing from the foregoing list of prior art skin-care products on towelettes are those products known as body lotions. More particularly, missing from the foregoing list of prior art skin care products are towelettes containing body lotion typically used by women who are particularly concerned about dry or rough skin or dry or rough skin patches which require the regular application of body lotion.

Because most people regularly use a number of different body lotions to treat a dry skin condition arising from a variety of factors including environmental conditions, and since there are no towelettes with body lotion presently available, there remains a need in the art for a towelette impregnated with body lotion. The body lotion must be formulated so as to provide all of the necessary materials to protect the skin that usually are found in more than one bottle of lotion.

### SUMMARY

According to the present invention, there is provided a towelette impregnated with body lotion.

The towelette itself that is impregnated with body lotion may be made from a variety of different natural and synthetic fibers.

The body lotion itself may include a variety of additives to enhance absorption by the skin, or other additives such as antioxidants, humectants, preservatives, and fragrances.

The towelette, after impregnation with body lotion, is enclosed within a leak proof packet.

The product, as seen by the consumer, is a foil packet with a towelette enclosed therein.

A typical packet containing a towelette impregnated with body lotion is about 3.5 inches square. Such sized packets may be easily stored in a suitcase, a purse, a back pack, an athletic equipment bag, a vehicle glove compartment, a drawer, etc. One reasonably skilled in the art will understand that many sizes of packet are enabled by this disclosure and are meant to be included in this disclosure.

Use of the body lotion towelette product of the present invention simply involves tearing open the packet, removing the towelette, and wiping the body lotion on a desired area of dry or rough skin. There is no waste or spillage of the body lotion, and the empty foil packet provides a neat and effective way of disposing of the used towelette.

### BRIEF DESCRIPTION OF THE DRAWING/FIGURES

A still better understanding of the invention disclosed in the present application my be had by examination of the drawing/figures wherein:

FIG. 1 is a perspective view of a towelette partially removed from a foil packet;

FIG. 2 is a flow chart of the steps involved in making/using the disclosed invention;

FIG. 3 is a flow chart of the process for making the body lotion with which the towelettes are impregnated; and

FIG. 4 is a flow chart of the steps involved in producing the disclosed invention.

### DESCRIPTION OF THE EMBODIMENTS

The ability to apply moisturizer to one's skin while away from home is challenged by the need to carry different bottles and containers containing various body moisturizing lotions. Carrying bulky containers is not only cumbersome but strenous as well. The natural inclination in such a situation is to avoid carrying the necessary body lotions. The result is damaged skin. As shown in FIG. 1, the preferred embodiment of the invention described in the instant application solves this problem by incorporating body lotion 20 into a small swatch of fabric or a towelette 10 which is enclosed in a leak proof aluminum foil-like plastic packet 30, which consists of two sides 31, a bottom 32 and a top 33. Such packet fits conveniently in one's pocket, purse, suitcase, briefcase or gym bag, for example.

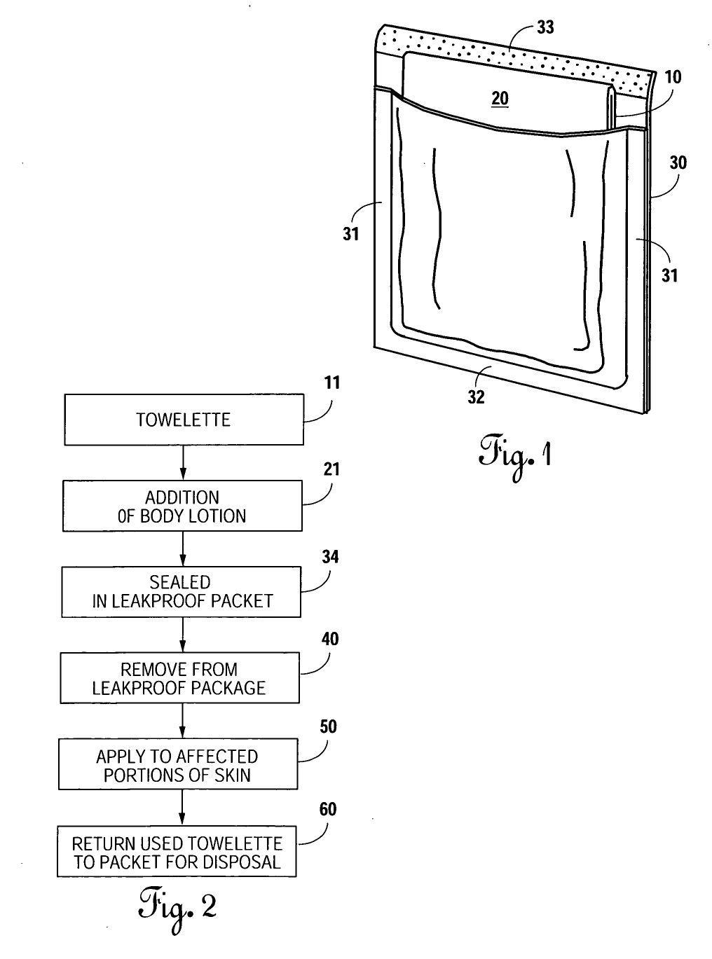

The disclosed invention is used by following some simple steps. As shown in FIG. 2, first, an 8″×8″ swath fabric or towelette 11 is moistened with body lotion 21. Next the towelette 11 impregnated with body lotion 20 is sealed in a leak proof packet 34. When needed, the towelette 11 is removed from the leak proof packet 40 and body lotion 20 is applied to the rough or dry portions of the skin 50. When the application of body lotion 20 is complete, the used towelette may be returned to the packet for disposal 60.

The towelette 10 could be made from natural, synthetic or semi-synthetic fibers. Exemplary synthetic fibers include polypropylene or polyethylene terephalate, rayon, polyester, polypropylene, etc. Examples of semi-synthetic fibers which could be used as towel fabric include cellulose pulp, or wood pulp. An example of a natural fiber which could be used as towelette fabric is cotton. In the preferred embodiment the fabric or towelette 10 is white and has a fiber denier of approximately 18.9 microns with a fiber denier range of approximately 18-20 microns per square meter. The fabric from which the towelette 10 is made is nonwoven and ranges in weight from approximately 42.5 to approximately 50 grams per square yard. Examples of those fabrics which are nonwoven include those that are spunbond, spunlace, meltblown, hydroentangled, airlaid and carded.

In another embodiment, the fabric or towelette could be colored light blue, pink or yellow with or without additional ornamentation such as flowers or cuddly animals which make the towelettes more attractive for use on small children. Persons of ordinary skill in the art will recognize that a number of other embodiments including different colors, designs, or shapes or sizes of towelette are disclosed by the present invention. Those other embodiments would include, among others, the size of the fabric swatch used, the type of fabric used, the weight and porosity of fabric employed and the color and other artwork applied to the fabric.

A body lotion 20 with a cps viscosity measurement of about 2765 with an approximate range in viscosity of 1000 to 3000 cps as measured with a Brookfield Viscometer will adequately impregnate the fabric or towelette described in the instant application. A mass of about 0.1367 grams of body lotion 20 is sufficient to adequately moisten the towelette 10 in the preferred embodiment. Those of ordinary skill in the art will understand that about 0.1000 grams of body lotion to about 0.1500 grams of body lotion may be used without departing from the present invention. Studies have shown that the body lotion 20 is essentially free of bacteria even after several months of storage. This absence of bacteria prevents premature breakdown of the body lotion 20 as a result of bacterial metabolism and promotes a long shelf life for the body lotion 20.

The body lotion 20 is composed of four parts (A, B, C and D) by percent weight as follows;

Part A:
Purified water—66.6
Glycerin—5
Methyl paraben—0.2
Disodium EDTA.—0.12
Ascorbic acid—0.04
Citric acid—0.15

Part B:
Avocado oil—0.26
Almond oil—7
Safflower oil—6
Jojoba oil—5
Retimi palmitate—0.5
Tocopherol acetate 0.5
Stearic acid—4.7
Cetyl alcohol—1.6
Polysorbate 40-1
Propyl paraben—0.2

Part C:
Triethanoloamine—0.9

Part D:
Fragrance—0.2

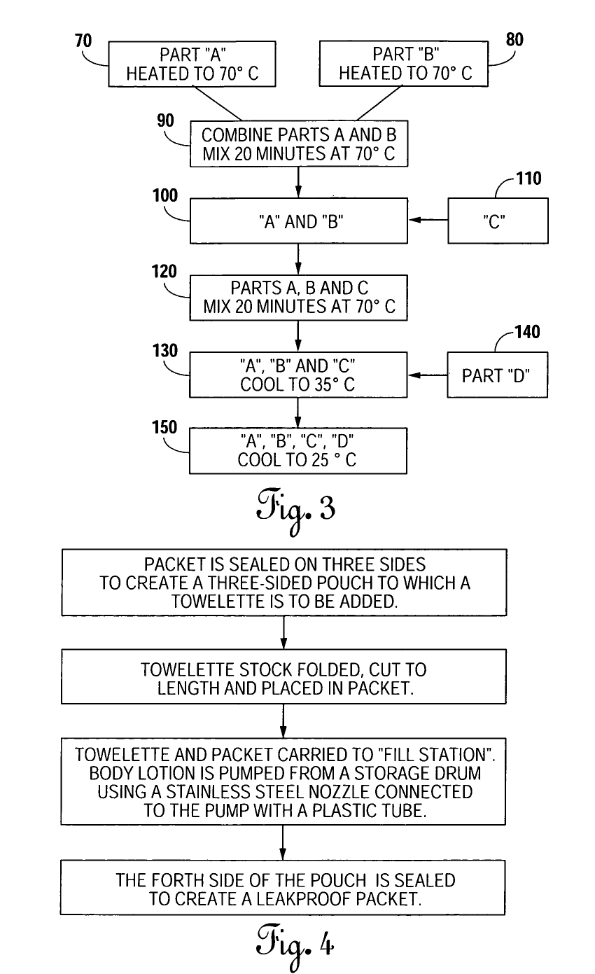

As shown in FIG. 3 Parts A, B, C and D are combined in the following manner:

Part A 70 and Part B 80 are heated to 70° C. separately. When Part A and Part B are at the desired temperature, Part A and Part B are combined 90, maintained at 70° C. and mixed for 20 minutes. Part C 110 is added to Parts A and B 100 and mixed for 20 minutes while maintaining the temperature at 70° C. 120. Allow the mixture of Parts A, B and C to cool to 35° C. 130 and add Part D 140. Mix Parts A, B, C, and D and cool to 25° C. with sweep agitation 150.

Referring again to FIG. 2, the towelette 10 which is inside the packet 30 which is closed on three sides is impregnated with body lotion 20. The fourth side of the packet 30 is then sealed to form a leak proof container for the towelette 10 which is impregnated with body lotion 20. The leak proof packet 30 is typically made from an aluminum, silver polyester laminate, or a polyester laminate with a foil lining. The materials used for the packet 30 need not only to be leak proof but are also constructed and sealed so as to prevent air and sunlight from entering the packet 30. Anti-oxidants added to the body lotion 20 degrade in the presence of air and sunlight and lose effectiveness in neutralizing oxidants and oxygen radicals on the skin. Such oxidants and oxygen radicals can be harmful to the skin.

When the towelette is needed, the leak proof packet 30 is opened 40 and the towelette is removed. The body lotion 20 is then spread over the desired area of skin 50. When finished, the used towelette is returned to the leak proof packet 60 providing a neat, mess-proof means of disposal.

The product is typically made according to the following steps as described in FIG. 4.

a) the packet is sealed on two sides 31 and the bottom 32 to create a three-sided pouch to which the towelette is added 16;

b) towel stock is folded, cut to length, and placed inside the packet.17;

c) the towelette 10 and the film are carried to a “fill station” 18;

d) body lotion 20 is pumped from a storage drum using a stainless steel pump 18 and is added to the towelette at the “fill station” using a stainless steel nozzle connected to the pump with a plastic tube;

e) the top, or fourth, side 33 of the foil pouch 30 is then sealed to create a leak proof packet 19;

f) a representative number of filled and sealed packets are weighed to assure that the proper amount of body lotion is added to the packets.

A person of ordinary skill in the art will recognize that there exist many other embodiments of the present invention which have not been specifically disclosed. It is the specific intent that these other embodiments be included within the scope and meaning of the appended claims.

What is claimed is:

1. A skin-care product comprising:
a towelette impregnated with body lotion;
said towelette being contained within a leak proof packet;
whereby access to body lotion on the towelette is gained by tearing open the leak proof packet, removing the towelette, and applying said body lotion impregnated towelette to the user's skin.

2. The skin-care product as defined in claim 1 wherein said towelette is made from unwoven fabric with a range in weight of about 42.5 to about 50 grams per square yard.

3. The skin-care product as defined in claim 1 wherein said body lotion includes one or more additives selected from a group including skin-absorption agents, antioxidants, humectants, preservatives, and fragrances.

4. The skin-care product as defined in claim 1 wherein said towelette is made from polypropylene with a fabric denier of about 18 to about 20 microns per square meter.

5. The skin-care product as defined in claim 1 wherein said towelette is made from polyethylene terephalate with a fiber denier of about 18 to about 20 microns per square meter.

6. The skin-care product as defined in claim 1 wherein said body lotion has a viscosity measurement with a range of approximately 1,000 to approximately 3,000 CPS.

7. The body lotion as defined in claim 1 wherein said body lotion is comprised of the following ingredients:
Purified water;
Glycerin;
Methyl paraben;
Disodium EDTA.;
Ascorbic acid;
Citric acid;
Avocado oil;
Almond oil;
Safflower oil;
Jojoba oil;
Retimi palmitate;
Tocopherol acetate;
Stearic acid;
Cetyl alcohol;
Polysorbate 40;
Propyl paraben;
Triethanolamine;
Fragrance.

8. The body lotion defined in claim 7 further including aloe.

9. The body lotion defined in claim 7 further including rooibos.

10. A method for making body lotion comprising the steps of
heating Part A to 70 C;
heating Part B to 70 C;
combining Part A and Part B;
mixing Part A and Part B for 20 minutes at 70 C;
adding Part C to the mixture of Parts A and B;
mixing Parts A, B and C for 20 minutes at 70 C;
cooling the mixture of Parts A, B and C to 35 C;
adding Part D to the mixture of Parts A, B and C;
mixing Parts A, B, C and D and cool to 25 C.

11. The method defined in claim 10 wherein Part A comprises:
Purified water;
Glycerin;
Methyl paraben;
Ddisodium EDTA.;
Ascorbic acid;
Citric acid.

12. The method defined in claim 10 wherein part B comprises:
Avocado oil;
Almond oil;
Safflower oil;
Jojoba oil;
Retimi palmitate;
Tocopherol acetate;
Stearic acid;
Cetyl alcohol;
Polysorbate;
Propyl paraben;

13. The method described in claim 10 wherein Part C is triethanolamine.

14. The method described in claim 10 wherein Part D is fragrance.

15. A method for making body lotion-impregnated towelettes comprising the steps of:
sealing the packet on three sides;
folding towelette stock;
cutting said towelette stock to length;
placing said folded and cut towelette into the packet material;
pumping body lotion into said packet from a storage drum using a stainless steel pump with a stainless steel nozzle connected to the pump by a length of plastic tubing;
sealing the fourth side of said packet to create a leak proof packet.

16. The method described in claim 15 wherein a step is included for:
weighing a representative number of filled and sealed said packets to control the amount of said body lotion added to each said packet.
